(19) United States
(12) Patent Application Publication
(10) Pub. No.: US 2007/0122854 A1
(43) Pub. Date: May 31, 2007

(54) Method for diagnosing colon cancer

(75) Inventor: Myeong-Jin Nam, Seoul (KR)

Correspondence Address: DARBY & DARBY P.C., P. O. BOX 5257, NEW YORK, NY, 10150-5257, US

(73) Assignees: RNL BIO CO. LTD., Seoul (KR); Myeong-Jin Nam, Seoul (KR)

(21) Appl. No.: 11/291,147

(22) Filed: Nov. 29, 2005

(51) Int. Cl. G01N 33/574; C12M 3/00

(52) U.S. Cl. 435/7.23; 435/287.2; 977/902

(57) ABSTRACT

The present invention relates to a method for diagnosing colon cancer by detecting a colon cancer specific antigen, defensin α6 from the blood of patient and a diagnostic kit for colon cancer comprising anti-defensin α6 antibody, The diagnostic kit for colon cancer of present invention comprises, a solid support such as 96-well plate for ELISA, nitrocellulose membrane, polyvinylidene fluoride membrane, microplate, glass substrate, polystyrene substrate, silicone substrate or metalplate, on which anti-defensin α6 antibody is immobilized; and, a means for detecting colon cancer specific antigen such as a primary antibody which specifically binds with an antigen conjugated with an antibody on a solid substrate and a secondary antibody-signal complex which specifically binds with the primary antibody. The diagnostic kit of the invention can diagnose colon cancer with the minute amount of patients' blood, which makes possible the easy and simple diagnosis of colon cancer.

### FIELD OF THE INVENTION

The present invention relates to a method for diagnosing colon cancer and a diagnostic kit therefor, more specifically, to a method for diagnosing colon cancer by detecting a colon cancer specific antigen, defensin α6 from the blood of patient and a diagnostic kit for colon cancer comprising anti-defensin α6 antibody.

### BACKGROUND OF THE INVENTION

Colorectal cancer is the second leading cause of cancer death in the United States, with 135,000 new cases diagnosed each year and an overall 5-year survival rate of ˜50%. Most colorectal. cancers develop slowly, beginning as small benign colorectal adenomas that progress over several decades to larger and more dysplastic lesions, eventually becoming malignant. This gradual progression provides ample opportunity for prevention and intervention. Diagnostic screening methods are at present suboptimal; therefore, new approaches are needed.

Early examination for colon cancer is generally performed with the digital rectal examination, sigmoidoscopy, colonoscopy or barium enema.

Digital rectal examination is made to check the normality of colon by an examiner wearing a glove and putting on some lubricant and inserting a hand into the rectum followed by the palpation, sigmoidoscopy is to observe the rectum and colon directly through a long and flexible mirror-attached tube, colonoscopy is to examine inside of the colon directly through an endoscope, and barium enema is to examine the abnormality of the blood stream distributed in the colon after injecting a contrast medium followed by the computed tomography.

The above-mentioned examinations are methods simply to assess the abnormality of the colon and so more precise tissue examination should be followed if lesions of diseases are diagnosed by those methods. For the biopsy performed after identifying lesions of diseases, it is relatively more accurate, but the diagnosis, which accompanies with some pain to patients, is so inconvenient that the patients tend to hesitate to take examinations. Therefore, needs for developing an easy and simple method for diagnosing colon cancer has been continued in the art.

On the other hand, a diagnosis method using genes has been developed for the diseases like cancers occurring genetic mutation and a considerable accomplishment has been reported on lung cancer, liver cancer and stomach cancer, but there are currently not many results reported on colon cancer. The diagnosis using genes is generally performed by PCR using DNA extracted from the tissue suspicious of cancer or by gene expression analysis using CDNA microarray for which RNA is extracted from the tissue. The former is effective only for specific cancers such as chronic myelogenous leukemia(CML) or acute lymphocytic leukemia(ALL) mainly caused by chromosome translocation, and the latter has a drawback that the conventional cancer diagnosis methods such as tissue examination, endoscopy or CT(computerized tomography) scans should precede,

In order to overcome the drawback of the cancer diagnosis methods described above, a method with which detects cancer-specific antigen and diagnoses cancer has been developed. For example, carcinoembryogenic antigen (CEA) has been reported to be a cancer-specific antigen, since the level of CEA is increased in the patients of rectum-colon cancer, stomach cancer, breast cancer, lung cancer, ovary cancer, prostate cancer or pancreas cancer(see: Bowser-Finn, R. A., Kahan, L., Larson, F. C., Traver, M. I. (1986) Tumour Biol. 7, 343-52; Carpelan-Holmstrom, M. A., Haglund, C. B., and Roberts, P. J. (1996) Dis Colon Rectum 39, 799-05). However,even if the cancer patients are diagnosed using CEA, further examination should be made to determine the kind of cancer precisely, which plays a barrier to easy and practical application of CEA in the diagnosis of cancers, since the CEA is commonly expressed in several cancers described above.

In this connection, there are strong reasons for exploring and developing an easy and simple method for diagnosing colon cancer with a high reliability and validity.

### SUMMARY OF THE INVENTION

The inventor have made an effort to screen a colon cancer specific antigen and develop a method for diagnosing colon cancer using an antibody against the antigen with a high reliability and validity, and found that colon cancer can be diagnosed by detecting a colon cancer specific antigen, defensin α6 from the blood of colon cancer patients,

A primary object of the present invention is, therefore, to provide a method for diagnosing colon cancer by detecting a colon cancer specific antigen, defensin α6.

The other object of the invention is to provide a diagnostic kit for colon cancer comprising anti-defensin α6 antibody.

### BRIEF DESCRIPTION OF DRAWINGS

The above and the other objects and features of the present invention will become apparent from the following description given in conjunction with the accompanying drawing, in which:

FIG. 1 is a schematic diagram depicting an immunochromatograpy strip as a preferred embodiment of a diagnostic kit for colon cancer.

### DETAILED DESCRIPTION OF THE INVENTION

Human defensins comprise a family of closely related, cationic polypeptides 29-42 amino acids in length. The polypeptides contain 6 conserved cysteines linked in disulfide bonds that stabilize the molecules as triple-stranded amphiphilic β-sheet structures(see: Ganz, T and Lehrer, R. I. (1994) Curr, Opin. Immunol. 6, 584-589; Hill, P.C., Yee, J., Selsted, M.E., and Eisenberg, D. (1991) Science251, 1481-1485). Invitro, human defensins exhibit antimicrobial activity against some bacteria, fungi, enveloped viruses, and parasites. Two classes of human defensiris, termed “α-defensins” and “β-defensins”, have been identified that differ with respect to their localization and linkage of cysteine residues, precursor peptide structure, and pattern of tissue expression. Whereas β-defensins are most abundant in epithelial cells of the lung, skin, and urogenital tract, the a-defensins were first found in human polymorphonuclear leukocytes and intestinal Paneth cells(see: Ganz, T., Selsted, M. E., Sklarck, D., Harwig, S. S. L., Daher, K., Bainton, D. F., and Lebrer, R. I. (1985) J. Clin. Invest. 76, 1427-1435; Mallow, E. B., Harris, A., Salzman, N., Russell, J. P., DeBerardinis, R. J., 20 Ruchelli, Z., and Bevins, C. L. J. Biol. Chem. 271, 4038-4045).

Although defensins seem to have diverse functional activities in innate antimicrobial immunity, a few reports have also indicated the presence of several defensins in epithelial tumors. There is much interest in identification of circulating tumor-derived proteins that may serve as biomarkers for the early detection of colon cancer. The inventor demonstrated that: the defensin α6 protein is expressed at higher levels in serum from colon cancer patients relative to serum from non-cancer controls; as such, defensin α6 may have utility as a biomarker for colon cancer.

Then, in an effort to identity potential molecular markers of colorectal tumors, the inventor implemented an approach based on the analysis of mictoatray data for the identification of tumor proteins that may have utility as biomarkers in colon cancer.

Expression analysis of microarray data obtained from a variety of 283 tumors and normal tissues revealed that defensin α6 was maximally expressed in colon cancer.

The present inventor obtained tissues from patients suffering from colon cancer, brain cancer, ovary cancer and pancreas cancer, and, from the genes expressed in each of the tissues, screened genes encoding proteins which are expressed in the tissue of colon cancer patient only with no expression in the tissues of the other cancer patients and normal human, and which are secreted to blood of the patients.

From the colon cancer-specific genes, genes encoding extracellular proteins, X14253(TOGF1), L21998(Mucin 2) and U33317(defensin α6), were selected, and Western blot analysis for blood samples from colon cancer patient, normal human and other cancer patients was followed by using an antibody specifically conjugated with proteins expressed by the said genes, which shows that: defensi.n α6 was detected in the colon cancer patient's serum only; Mucin 2 was detected in the colon cancer patient's serum and other cancer patient's serum; and, TDGF 1 was not detected in the colon cancer patient's serum. Accordingly, it was found that defensin a 6 is a colon cancer-specific protein which can be used as a biomarker for diagnosing colon cancer.

A method for diagnosing colon cancer of present invention comprises a step of detecting defensin α6from the blood of patient; which is expressed in the colon cancer tissue and secreted to blood, where the detection method of defensin α6, not limited thereto, includes immune reaction with anti-defensin α6 antibody, chromatography and electrophoresis.

The method for diagnosing colon cancer of present invention can be practiced by employing a diagnostic kit comprising anti-defensin α6 antibody. Preferably, the diagnostic kit for colon cancer comprises anti-defensin α6 antibody immobilized on a solid support and a means for detecting colon cancer specific antigen conjugated with the said antibody.

The solid support, not limited thereto, includes 96-well plate for ELISA, nitrocellulose membrane, polyvinylidene fluoride membrane, microplate, glass substrate, polystyrene substrate, silicone substrate and metal plate.

The means for detecting colon cancer specific antigen, not limited thereto, includes a primary antibody-gold particle conjugate which specifically binds with an antigen conjugated with an antibody on the solid substrate and a primary antibody which specifically binds with an antigen conjugated with an antibody on a solid substrate and a secondary antibody-signal complex which specifically binds with the primary antibody.

The signal, not limited thereto, includes fluorescent material such as Cy-3, Cy-5, FITC(fluoroisothiocyanate), GFP(green fluorescent protein), RFP(red fluorescent protein) and Texas Red, the radioisotope, not limited thereto, includes ³²p, ³H and ¹⁴C, and the enzyme , not limited thereto, includes kRR(horse radish peroxidase), alkaline phoaphatase, β-galactosidase and luciferase, and in case of using the enzyme as the signal, the kit further comprises a substrate for the enzyme.

As a preferred embodiment of the invention, the kit for diagnosing colon cancer is provided as an ELISA kit comprising anti-defensin α6 antibody. Preferably, it comprises 96-well plate for ELISA coated with anti-defensin α6 antibody, and a means for detecting defensin α6 such as anti-defensin α6 antibody, and a secondary antibody-signal complex such as HRP(horseradish peroxidase)-conjugated goat anti-rabbit IgG antibody and TMB(tetramethyl benzidine) as a substrate for HRP.

As the other preferred embodiment of the invention, the kit for diagnosing colon cancer is provided as an immunochromatography strip comprising a membrane on which anti-defensin α6 antibody is immobilized, and a means for detecting defensin α6, i.e., a gold particle bound anti-defensin α6 antibody, where the membrane, not limited thereto, includes NC membrane and PVDF membrane.

Preferably, it comprises a plastic plate on which a sample application pad, a gold particle bound anti-defensin α6 antibody temporally immobilized on a glass fiber filter, a nitrocellulose membrane on which anti-defensin α6 antibody band and a secondary antibody band are immobilized and an absorbent pad are positioned in a serial manner, so as to keep continuous capillary flow of blood serum.

In case of using the kit as described above, colon cancer patient can be diagnosed by adding blood serum from patient to a diagnostic kit and detecting defensin α6 conjugated with anti-defensin α6 antibody, specifically, by a method which comprises the steps of: (i) collecting blood from the patient;

(ii) separating blood serum from the patient's blood; (iii) adding the blood serum from patient to a diagnostic kit; and, (iv) detecting defensin α6 conjugated with anti-defensin α6 antibody.

The diagnostic kit of the invention can diagnose colon cancer with the minute amount of patients' blood, which makes possible the easy and simple diagnosis of colon cancer.

The present invention is further illustrated in the following examples, which should not be taken to limit the scope of the invention.

### EXAMPLE 1

Screening of Colon Cancer-Specific Genes

The tissues analyzed were consisting of 6 normal brain (BN) 19 brain cancrs (SAI), 8 normal colon(CN), 38 microsatellite stability-type colon cancers(CMSS), 13 microsatellite instability-type colon cancers (CMSX), 10 normal lung (LN), 37 lung cancers(LI), 7 normal pancreas(PN), 8 pancreas cancers (PT). Tissues samples were homogenized in the presence of Trizol reagent (Life Technologies, Inc., USA) and total cellular RNA was purified according to manufacturer's procedures. RNA samples were further purified using RNeasy spin columns(Oiagen, USA). RNA quality of the lung and ovary tumors was assessed by 1% agarose gel electrophoresis in the presence of ethidium bromide. Samples that did not reveal intact and approximately equal 18S and 28S ribosomal bands were excluded from this experiment. This experiment used commercially available high-density microarrays (Affymetrix, USA) that produced gene expression levels on 7129 known genes and expressed sequence tags (HuGeneFL Array). Preparation of cRNA, hybridization, and scanning of the arrays were performed according to manufacturer's protocols.

Briefly, 5 μg of the total RNA was used to generate double-stranded CONA by reverse transcription using a cDNA synthesis kit (Superscript Choice System, Life Technologies, Inc, USA) that uses an oligo (dT)₂₄ primer containing a T7 RNApolymerase promotes 3′ to the polyT (Geneset, USA), followed by second-strand synthesis. Labeled cRNA was prepared from the double-stranded cDNA by in vitro transcription by T7RNA polymerase in the presence of biotin-11-CTP and biotin-16-UTP(Enzo, USA). The labeled cRNA was purified over RNeasy columns. 151ig of cRNA was fragmented at 94° C. for 35minutes in 40 mmol/Lof Tris-acetate, pH8.1, 100 nmmol/L of potassium acetate, and 30 mmol/L of magnesium acetate. The cRNA was then used to prepare 300 μl of hybridization cocktail(100 mmol/L MES, 1 mol/L NaCl, 20 mmol/L ethylenediaminetetraacetic acid, 0.01% Tween 20) containing 0.1 mg/ml of herring sperm DNA (Promega, USA) and 500μ /ml of acetylated bovine serum albumin (Life Technologies, Inc., USA). Before hybridization, the cocktails were heated to 94° C. for 5 minutes, equilibrated at 45° C. for 5 minutes, and then clarified by centrifugation(16,000 ×g) at room temperature for 5 minutes. Aliquots of this hybridization cocktail containing 10μg of fragmented CRNA were hybridized to HuGeneFL arrays at 45° C. for 16 hours in a rotisserie oven for 60 rpm. The arrays were washed using nonstringent buffer (6×SSPE) at 25° C., followed by stringent buffer (100 mmol/L MES, pH 6,7, 0.1 mol/L NaCl, 0.01% Tween 20) at 50° C.

The arrays were stained with streptavidin-phycoerythrin (Molecular Probes, USA), washed with 6× sodium chloride, sodium phosphate, EDTA(SSPE buffer), incubated with biotinylated anti-streptavidin IgG, stained again with streptavidin-phycoerythrin, and washed again with 6×SSPE. The arrays were scanned using the GeneArray scanner (Affymetrix, USA). Image analysis was performed with GeneChip software (Affymetrix, USA), and colon cancer-specific genes expressed in colon cancer tissues were selected by way of quantile-normalization (see: Table 1). As can be seen in Table 1 below, described genes were expressed highly in colon cancer tissues than normal colon as well as other tissues.

TABLE 1
Comparison of expression Level of Colon Cancer-Specific
Genes in Various Cancer Tissues

| | Tissue* | | | | | | | | |
|---|---|---|---|---|---|---|---|---|---|
| Gene** | BN | BAI | CN | CMSS | CMSI | LN | LI | PN | PT |
| U51096 | −19 | 16 | 111 | 1086 | 805 | 21 | 31 | 57 | 31 |
| U22376 | 42 | −9 | −19 | 1956 | 1195 | 127 | 149 | 56 | -31 |
| U51095 | 33 | 93 | 193 | 2474 | 2564 | 58 | 51 | 438 | 179 |
| X14253 | 58 | 65 | 85 | 1349 | 405 | 22 | 57 | 236 | 80 |
| L21998 | 859 | 1037 | 245 | 6139 | 9306 | 893 | 1141 | 1345 | 1057 |
| U30246 | 93 | 29 | 288 | 1374 | 2160 | 70 | 151 | 108 | 80 |
| U33317 | 171 | 149 | 405 | 1494 | 1058 | 59 | 134 | 76 | 31 |
| U79725 | 65 | 26 | 730 | 2622 | 1862 | 345 | 102 | 10 | 49 |
| L02785 | 68 | 67 | 337 | 1710 | 108 | 13 | 32 | 87 | 213 |
| M10050 | −91 | −135 | 2652 | 9101 | 2416 | −148 | −81 | −12 | −68 |
| X83228 | −35 | −27 | 626 | 4641 | 3389 | −45 | 41 | 56 | 147 |
| M30496 | 356 | 341 | 290 | 1954 | 1273 | 382 | 435 | 189 | 478 |
| U26726 | 91 | 43 | 463 | 1882 | 1464 | 97 | 237 | 309 | 347 |
| X12901 | −21 | 13 | 188 | 1578 | 791 | −44 | 183 | 205 | 141 |
| M73489 | 204 | 169 | 362 | 1553 | 987 | 106 | 172 | 686 | 168 |
| M76180 | 6 | 14 | 647 | 2615 | 1300 | 116 | 326 | 580 | 189 |
| L25931 | 166 | 571 | 386 | 2177 | 1665 | 618 | 702 | 341 | 483 |
| U55206 | 117 | 162 | 159 | 2311 | 1149 | 141 | 292 | 38 | 124 |
| M30703 | −26 | 64 | 102 | 1214 | 595 | 487 | 537 | 22 | 239 |
| *BN, normal brain tissue; BAI, brain cancer tissue; CN, normal colon tissue, CMSS, MSS (microsatellite stability-type) colon cancer tissue, CMSI, MSI (microsatellite instability-type) colon cancer tissue; LN, normal lung tissue; LI, lung cancer tissue; PN, normal pancreas tissue; PT, pancreas cancer tissue | | | | | | | | | |
| **The proteins encoded in colon cancer-specific genes are obtained from the manual of Microarray (HuGeneFL Array, Affmetrix, USA): U51096, caudal type homeo box transcription factor 2; U22376, avian myeloblastosis viral oncogene homolog; U51095, caudal type homeo box transcription factor 1; Xl4253, teratocarcinoma-derived growth factor 1: TDGFl; L21998, intestinal/tracheal mucin 2; U30246, sodium/potassium/chloride transporter; U33317, | | | | | | | | | |
| # Paneth cell-specific defensin α6; U79725, trans-membrane glycoprotein A33; L02785, solute carrier family 26, member 3; M10050, fatty acid binding protein 1; X83228, cadherin 17 as liver-intestine cadhedrine; M30496, ubiquitin carboxyl-terminal osterase L3 as ubiquitin thiolesterase; U26726, hydroxysteroid (11-beta) dehydrogenase 2; X12901, villin 1; M73489, guanylate cyclase 2C as heat stable enterotoxin receptor; M76180, DOPA decarboxylase | | | | | | | | | |
| # as aromatic L-amino acid decarboxylase: L25931, lamin B receptor, U55206, gamma-glutamyl hydrolase as folylpolygamma-glutamyl hydrolase; M30703, amphiregulin as schwannoma-derived growth factor. | | | | | | | | | |

### Example 2

Western Blot Analysis

From colon cancer-specific genes in Example 1, genes encoding extracellular proteins, X14253(TDGF1), L21998(Mucin 2) and U33317(defensin α6), were selected, and Western blot analysis for blood samples from a colon cancer patient, normal human and other cancer patient was accomplished by using an antibody specifically conjugated with proteins expressed by the said genes as follows:

Serum was first separated from the blood of patients, who donated normal and cancer tissues in Example 1, and subjected to 10% SDS-polyacrylamide gel electrophoresis, and, then transferred to PVDF membrane by electrical means. The PVDF membrane was blocked by incubation for 12-14hours in a blocking buffer(3% bovine serum albumin, 0.05% Tween 20 in PBS) to reduce non-specific binding of proteins transferred to the membrane. Subsequently, the blocked PVDF membranes were incubated for 2hours at room temperature with anti-TDGF 1 rabbit polyclonal antibody(Siocat, Germany), anti-Mucin 2 rabbit polyclonal antibody(Abeam, UK) and anti-defensin α6 rabbit polyclonal antibody(Alpha Diagnostic International, USA) at a 1:200 (v/v) dilution in the blocking buffer, The membranes was washed in PBST(0.05% Tween 20 in PBS) for 10 minutes three times, and incubated for 45 minutes with horseradish peroxidase-conjugated goat anti-rabbit IgG antibodies(Santa Cruz Biotechnology, USA) at a 1:200 (v/v) dilution in the blocking buffer, and then washed in PBS for 10 minutes three times. Immunodetection was accomplished by enhanced chemiluminescence(ECL™ kit, Amersharn, UK), followed by PhosphaImager (Fuji, Japan).

As a result, defensin α6 and Mucin 2 were detected in the colon cancer patient's serum, while TDGF 1 was not detected, indicating that defensin α6 is a colon cancer-specific protein which can be used as a biomarker for diagnosing colon cancer.

### Example 3

Preparation of ELISA Kit for Diagnosing Colon Cancer and Analysis of Blood Sample Using the Kit

### Example 3-1

Preparation of ELISA Kit for Diagnosing Colon Cancer

An ELISA kit for diagnosing colon cancer was prepared to comprise a 96-well plate for ELISA, anti-defensin α6 rabbit polyclonal antibody, HRP(horseradish peroxidase)-conjugated goat anti-rabbit IgG antibody and TMB(tetramethyl benzidine).

A 96-well maxi-sorp microtiter plate (Nunc, USA) was coated by incubation overnight at 4° C. with anti-defensin α6 rabbit polyclonal antibody (50 μl /well) at a 1:50 (v/v) dilution in a coating buffer (Na₂CO₃ 0.188% (w/v), NaNCO₃ 0.271% (w/v), NaCl 0.731% (w/v), pH 9.6). After washing in PBS three times, the coated wells were blocked by incubation for 1 hour at 4° C. in a blocking buffer (3% (w/v) BSA, 0.05% (w/v) Tween 20 in PBS) to block non-specific binding, and dried and sealed up to prepare a 96-well plate for ELISA kit.

Then, commercially available anti-defensin α6 antibody, HRP(horseradish peroxidase)-conjugated goat anti-rabbit IgG antibody(Alpha Diagnostic International, USA) and TMB(tetramethyl benzidine) (Sigma Chem. Co., USA), all of which are used together with the 96-well plate for ELISA kit.

### Example 3-2

Analysis of Blood Sample 200 μl of serum of a colon cancer patient, pacreas cancer patient, ovary cancer patient, brain cancer patient and normal human were added to each well of ELISA plate prepared in Example 3-1. After the first antigen-antibody interaction was accomplished by mild shaking the plate for 1 hour at room temperature, the serum Was removed from each well, and the wells were then washed five times in PBS.

Subsequently, 50 μl of the anti-defensin α6 polyclonal antibody at a 1:200 (v/v) dilution in PBST was added to each well in which the primary antigen-antibody interaction was accomplished, and the second antigen-antibody interaction was accomplished for 2 hours at room temperature, and the wells were then washed three times in PBST. The wells were then blocked by incubation for 1 hour at room temperature to block non-specific conjugation. And then, 50 μl of HRP-conjugated goat anti-rabbit IgG at a 1:400 dilution in the blocking buffer was added to each well in which the secondary antigen-antibody interaction was accomplished, and incubated by slow shaking for 2hours at room temperature,

The said plate was washed four times in PBST, and a coloring agent, TMB(tetramethyl benzidine, Sigma Chem. Co., USA) was added to the plate at a concentration according to manufacturer's protocols, and then added 1.25M H₂SO₄ to stop the color reaction. Subsequently, the wells were read at 450 nm using a microtiter plate reader(Model 680 Microplate Reader, Biorad, USA) (see: Table 2).

TABLE 2
ELISA Results of Serum Samples from Normal Human and
Patients Suffering from Various Cancers (unit: absorbance
at 450 nm)

| Cancer-specific | Normal | Colon | Pancreas | Ovary | Brain |
|---|---|---|---|---|---|
| Antigen | human | cancer | cancer | cancer | cancer |
| Defensin α6 | 0.083 ± 0.016 | 0.251 ± 0.039 | 0.075 ± 0.021 | 0.078 ± 0.024 | 0.091 ± 0.031 |

As can be seen in Table 2 above, it was clearly demonstrated that the level of defensin α6 in colon cancer patient was higher more than double than that in normal human and other cancers patients, indicating that the ELISA kit can be used for the diagnosis of colon cancer with a high reliability and validity.

### Example 4

Preparation of Immunochromatography Strip for Diagnosing Colon Cancer and Analysis of Blood Sample

### Example 4-1

Preparation of Sample Application Pad

Cellulose filter(Millipore, USA) was cut in a size of 0.8×1.2 cm to prepare a sample application pad for immunochromatography strip.

### Example 4-2

Preparation of Immune Reaction Member

A gold particle bound anti-defensin α6 antibody was immobilized on a glass fiber (GF) filter temporally to prepare an immune reaction member: AuCl (gold monochloride) was treated with sodium citrate solution to give a solution containing reduced gold particle with a particle size of 40 nm having an optical density of 10±1 at 532 nm. To AuCl solution was added anti-defensin a 6 antibody at a concentration of 10 μg/ml and added PEG (polyethylene glycol)to obtain a primary antibody-gold particle conjugate solution. Subsequently, the glass fiber. filter (0.8 cm×1.0 cm, Millipore, USA) was impregnated with the antibody-gold particle conjugate solution and dried at 37° C., finally to prepare an immune reaction member.

### Example 4-3

Preparation of Result Indicator NC membrane was cut to have a size of 0.8 cm×5 cm,and a decision line was created in a position of 0.8 cm apart from the lower end of the membrane in the length direction and the control line was created in a position of 0.8 cm apart from the decision line in the upper direction, to give a result indicator in a form of NC membrane. The decision line was created in a straight line using a mixture of anti-defensin α6 antibody and PBST at a 1:50 (v/v) dilution and dried at 37° C., and the control line was created in a straight line using a mixture of goat anti-rabbit IgG (Santa-Cruz, USA) and PBST at a 1:50 (v/v) dilution and dried at 37° C.

### Example 4-4

Preparation of Absorbent Pad

A cellulose filter (Millipore, USA) was cut to have a size of 0.8 cm×3 cm to prepare an absorbent pad, which allows continuous capillary flow of blood sample by absorbing non-reactive materials after immune reaction.

### Example 4-5

Preparation of Immunochromatography Strip for Diagnosing Colon Cancer

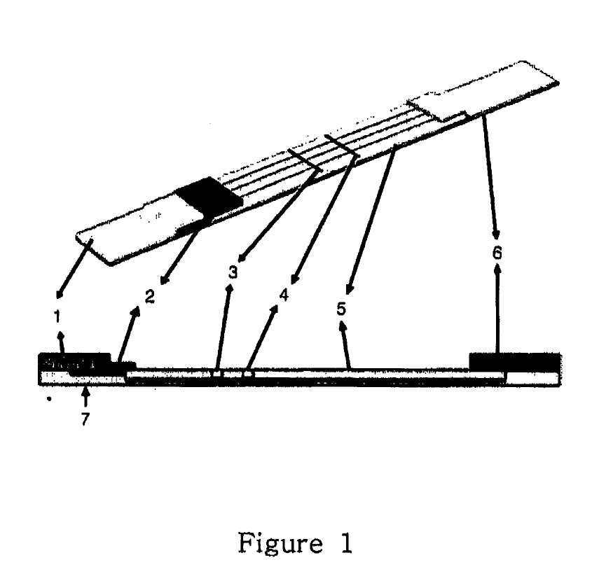

On a plastic plate having a size of 0.8 cm×12 cm (Millipore, USA), the sample application pad prepared in Example 4-1, the immune reaction member prepared in Example 4-2, the result indicator prepared in Example 4-3 and the absorbent pad prepared in Example 4-4 were positioned serially in the length direction, to prepare an immunochromatography strip for diagnosing colon cancer. The sample application pad, immune reaction member, result indicator and absorbent pad are positioned, in a manner that the said components overlapped one another to keep continuous capillary flow of liquid sample. FIG. 1 is a schematic diagram depicting an immunochromatograpy strip for colon cancer, which comprises a plastic plate (7) on which a sample application pad (1), a gold particle bound anti-defensin α6 antibody temporally immobilized on a glass fiber filter(2), a nitrocellulose membrane (S) on which anti-defensin α6 antibody band (3) and a secondary antibody band (4) are immobilized and an absorbent pad(6) are positioned in a serial manner, so as to keep continuous capillary flow of blood serum.

### Example 4-6

Analysis of Blood Samples

Blood samples were collected from 5 normal humans, 7 colon cancer patients, 5 pancreas cancer patients, 4 ovary cancer patients and 3 brain cancer patients, and 3 ml of each blood samples was added to the sample application pad of the immunochromatography strip prepared in Example 4-5. After five minutes, it was examined whether gold particle conjugates in decision line were detected or not. As a result, it was found that only in the blood samples of colon cancer patients, gold particle conjugates were detected in decision line.

Therefore, the immunochromatography strip of the invention can diagnose colon cancer, which makes possible the easy diagnosis of colon cancer with a high reliability and validity.

As clearly illustrated and demonstrated as above, the present invention provides a method for diagnosing colon cancer by detecting a colon cancer specific antigen, defensin α6 from the blood of patient and a diagnostic kit therefor, The diagnostic kit of the invention can diagnose colon cancer with the minute amount of patients' blood, which makes possible the easy and simple diagnosis of colon cancer.

1. A method for diagnosing colon cancer which comprises a step of detecting defensin α6 from blood of patient.

2. The method for diagnosing colon cancer of claim 1, wherein the defensin α6 is detected by way of immune reaction with anti-defensin α6 antibody, chromatography or electrophoresis.

3. A diagnostic kit for colon cancer which comprises anti-defensin α6 antibody immobilized on a solid support and a means for detecting colon cancer specific antigen conjugated with the said antibody.

4. The diagnostic kit for colon cancer of claim 3, wherein the solid support is 96-well plate for ELISA, nitrocellulose membrane, polyvinylidene fluoride membrane, microplate, glass substrate, polystyrene substrate, silicone substrate or metal plate.

5. The diagnostic kit for colon cancer of claim 3, wherein the means for detecting colon cancer specific antigen is a primary antibody-gold particle conjugate which specifically binds with an antigen conjugated with an antibody on a solid substrate.

6. The diagnostic kit for colon cancer of claim 3, wherein the means for detecting colon cancer specific antigen comprises a primary antibody which specifically binds with an antigen conjugated with an antibody on a solid substrate and a secondary antibody-signal complex which specifically binds with the primary antibody.

7. The diagnostic kit for colon cancer of claim 6, wherein the signal is fluorescent material, radioisotope or enzyme.

8. The diagnostic kit for colon cancer of claim 7, wherein the fluorescent material is Cy-3, Cy-5, FITC (fluoroisothiocyanate), GFP (green fluorescent protein), RFP (red fluorescent protein) or Texas Red.

9. The diagnostic kit for colon cancer of claim 7, wherein the enzyme is HRP (horseradish peroxidase), alkaline phosphatase, β-galactosidase or luciferase.

10. An ELISA kit for diagnosing colon cancer which comprises a 96 well plate for ELISA coated with anti-defensin α6 antibody, anti-defensin α6 antibody, HRP (horseradish peroxidase)-conjugated goat anti-rabbit IgG antibody and TMB (tetramethyl benzidine).

11. An immunochromatography strip for diagnosing colon cancer comprising a membrane on which anti-defensin α6 antibody is immobilized, and a means for detecting defensin α6.

12. The immunochromatography strip for diagnosing colon cancer of claim 11, wherein the membrane is NC membrane or PVDF membrane.

13. The immunochromatography strip for diagnosing colon cancer of claim 11, wherein the means for detecting defensin α6 is a gold particle bound anti-defensin α6 antibody.

14. An immunochromatography strip for diagnosing colon cancer comprising a plastic plate on which a sample application pad, a gold particle bound anti-defensin α6 antibody temporally immobilized on a glass fiber filter, a nitrocellulose membrane on which anti-defensin α6 antibody band and secondary antibody band are immobilized and an absorbent pad are positioned in a serial manner, so as to keep continuous capillary flow of blood serum.

15. A method for diagnosing colon cancer which comprises the steps of adding blood serum from patient to a diagnostic kit described in claim 3 and detecting defensin α6 conjugated with anti-defensin α6 antibody.
